(19) United States
(12) Patent Application Publication
(10) Pub. No.: US 2007/0269533 A1
(43) Pub. Date: Nov. 22, 2007

(54) Oral Dieting Composition Comprising Conjugated Linoleic Acid and Caffeine

(75) Inventor: Bernadette Verneau, Frejus (FR)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: INSTITUTE PHYTOCEUTIC, Frejus France (FR)

(21) Appl. No.: 10/587,441

(22) PCT Filed: Jan. 26, 2005
(86) PCT No.: PCT/FR05/00166

§ 371 Date: Apr. 4, 2007

(30) Foreign Application Priority Data
Jan. 27, 2004 (FR) 0400739

(51) Int. Cl. A61K 31/522; A61K 33/00; A61K 33/24; A61P 3/04

(52) U.S. Cl. 424/655; 424/724; 514/263.31

(57) ABSTRACT

The invention relates to a dieting composition for oral administration, comprising a mixture of conjugated linoleic acid and caffeine as active ingredient and an acceptable support for oral administration.

The present invention relates to a slimming composition for administration by the oral route, comprising a combination of conjugated linoleic acid and caffeine as active ingredient.

Conjugated linoleic acid (CLA) is a mixture of isomers of linoleic acid which are naturally present in milk and dairy products, and in the meat of ruminants. The term CLA includes all the configuration and position isomers of octadecadienoic acid (C18:2), in particular the cis and trans forms of 9,11-, 10,12- and 11,13-octadecadienoic acids.

It has been demonstrated that the oral administration of CLA makes it possible to reduce the fatty mass (stored fat) in favor of the lean mass (muscles), both in humans and in animals for slaughter. The effective dose is from 1 to 2 g/day.

Two mechanisms are thought to explain this advantageous activity. On the one hand, the CLA modifies the metabolism of the lipids and in particular reduces their storage in the form of fat. On the other hand, CLA increases the energy expenditure in the body (or thermogenesis) and thus promotes the use of reserve fat as source of energy.

Caffeine is an alkaloid which is present in numerous plants, in particular in the beans of the coffee tree, the leaves of the tea plant or the nuts of the kola tree. In addition to its psychological and diuretic stimulant activity, caffeine is also known as an agent stimulating thermogenesis and therefore as a slimming agent by the oral route.

Now, the applicant has discovered quite unexpectedly that the combination of CLA and caffeine, administered by the oral route, makes it possible to stimulate even more these mechanisms for elimination of stored fat which lead to loss of weight. It has been demonstrated that CLA and caffeine act in synergy.

The subject of the present invention is a slimming composition for administration by the oral route, comprising:
a combination of conjugated linoleic acid and caffeine as active ingredient, and
an acceptable carrier for administration by the oral route.

The invention relates more particularly to a slimming composition for administration by the oral route, comprising:
a combination of conjugated linoleic acid and caffeine as active ingredient with a conjugated linoleic acid/caffeine mass ratio of between 1 and 15, preferably between 1 and 6, and
an acceptable carrier for administration by the oral route.

The acceptable carrier for administration by the oral route may be any physiologically acceptable liquid, solid or pasty substance, which is inert to a greater or lesser degree and into which said active ingredient is incorporated in order to facilitate the preparation and the oral administration thereof and to determine the consistency, the form and the volume thereof.

This carrier may be in particular a dietary carrier. Thus, said composition may be advantageously provided in the form of soluble coffee, that is to say spray-dried or freeze-dried powdered coffee which is soluble in water and which is obtained exclusively from roast coffee by physical methods using water as sole carrying agent not obtained from the coffee.

Indeed, the most common source of caffeine is coffee which is generally consumed in the form of a drink. The caffeine content can vary widely according to the type of coffee and the mode of preparation. The table which follows gives nevertheless an approximate order of magnitude for a cup of 237 ml. This table also makes it possible to observe that the caffeine may come from other drinks such as tea or cola-based fizzy drinks, but that their caffeine content is less than that of coffee.

| Type of drink | Caffeine content | |
|---|---|---|
| filtered coffee | 179 | mg |
| infusion coffee | 135 | mg |
| percolator coffee | 118 | mg |
| instant coffee | From 75 mg to 106 mg | |
| tea | 30 to 50 | mg |
| cola-type fizzy drink | 36 to 50 | mg |

Caffeine makes it possible to stimulate thermogenesis, but only at high doses, in particular at doses greater than 600 mg/day. Now, at a high dose, caffeine can cause undesirable effects such as insomnia, irritability, tachycardia or hypertension, or may even be toxic. If CLA is combined with caffeine, then the daily dose required to obtain a slimming effect is reduced or for the same daily dose, the slimming effect of coffee is increased.

However, CLA exists in the form of an oil which is difficult to disperse in water. If CLA is added to liquid coffee, oil drops form at the surface of the liquid, which is not satisfactory for the consumer. To produce a coffee formulation supplemented with a large quantity of CLA and which then allows perfect dispersion of the CLA in the reconstituted liquid coffee, it is possible to combine CLA with lecithin and colloidal silica. These two adjuvants indeed make it possible to prepare CLA in the form of a powder which is perfectly dispersed in an aqueous medium.

Said composition can therefore be provided in the form of a powder, tablets, gelatin capsules, capsules or sachets of powder.

Said composition advantageously contains lecithin and colloidal silica to allow good dispersion of the CLA in an aqueous medium.

Said composition may also contain other active ingredients which help to limit the level of carbohydrates in the blood, such as a green coffee extract preferably containing from 5 to 10% by weight of caffeine, and/or chromium chloride.

Said composition may be used as a dietary supplement, a dietetic composition or a cosmetic composition, in particular to increase weight loss, in the context of an esthetic treatment designed to improve the figure of a person.

Said composition may also be used as a medicament, in particular intended to treat or prevent obesity. The therapeutic objective in the case of obesity is defined in the following manner: the aim is either to allow the subject to lose weight significantly, or to help the subject to retain a weight that is as low as possible.

The effective dose for increasing weight loss is at least 60 mg of caffeine and 300 mg of CLA, twice per day.

### EXAMPLE OF FORMULATION

### 1 Sachet

| | |
|---|---|
| spray-dried coffee (containing 60 mg of caffeine): | 2390 mg |
| conjugated linoleic acid: | 310 mg |
| lecithin: | 30 mg |
| green coffee extract (containing 5% by weight of | 240 mg |
| caffeine): | |
| chromium chloride (containing 12.5 μg of chromium): | 0.064 mg |
| colloidal silica: | 30 mg |

Eight volunteers took this formulation by the oral route in an amount of 2 sachets/day for one month, without changing their dietary habits, and each lost on average more than 4 kg.

An additional study was carried out on 24 people with excess weight, to whom the formulations of the following table were administered by the oral route, in an amount of two sachets/day for one month, without changing their dietary habits.

| | | Weight loss |
|---|---|---|
| | | observed |
| Formulation tested (1 sachet) | | (as % by weight) |
| Spray-dried coffee (containing | 2390 mg | −0.6 (N.S.) |
| 60 mg of caffeine) | | |
| Conjugated linoleic acid | 310 mg | −1.8 (N.S.) |
| Spray-dried coffee (containing | 2390 mg | −4.2 (p < 0.05) |
| 60 mg of caffeine) | | |
| Conjugated linoleic acid | 310 mg | |
| Spray-dried coffee (containing | 2390 mg | −4.8 (p < 0.01) |
| 60 mg of caffeine) | | |
| Conjugated linoleic acid | 310 mg | |
| Green coffee extract | 240 mg | |
| Chromium chloride (containing | 0.064 mg | |
| 12.5 μg of chromium) | | |

The weight loss observed for coffee alone or CLA alone is not significant. On the other hand, it is significantly increased when the CLA and the caffeine are combined in the same formulation.

These results confirm the synergy between caffeine and conjugated linoleic acid on weight loss.

1. A slimming composition for administration by the oral route, comprising:
a combination of conjugated linoleic acid and caffeine as active ingredient, and
an acceptable carrier for administration by the oral route, in which the conjugated linoleic acid/caffeine mass ratio is between 1 and 15.

2. The composition as claimed in claim 1, wherein the conjugated linoleic acid/caffeine mass ratio is between 1 and 6.

3. The composition as claimed in claim 1, wherein it additionally contains lecithin and colloidal silica.

4. The composition as claimed in claim 1, wherein it additionally contains a green coffee extract, and/or chromium chloride.

5. The composition as claimed in claim 1, wherein it is provided in powdered form or in liquid form.

6. The composition as claimed in claim 5, wherein it is provided in the form of soluble coffee.

7. The composition as claimed in claim 1, wherein it is provided in the form of tablets, gelatin capsules, capsules or sachets of powder.

8. The composition as claimed in claim 6, wherein it contains:

| | |
|---|---|
| spray-dried coffee (containing 60 mg of caffeine): | 2390 mg |
| conjugated linoleic acid: | 310 mg |
| lecithin: | 30 mg |
| green coffee extract (containing 5% by weight of caffeine): | 240 mg |
| chromium chloride (containing 12.5 μg of chromium): | 0.064 mg |
| colloidal silica: | 30 mg |

9. The composition as claimed in claim 1, as a dietary supplement, a dietetic composition or a cosmetic composition.

10. A method for increasing weight loss in a person comprising administering to said person by oral route the composition as claimed in claim 1.

11. (canceled)

12. A method for treating or preventing obesity in a patient comprising administration to said patient by oral route the composition as claimed in claim 1.
